(19) United States
(12) Patent Application Publication
(10) Pub. No.: US 2007/0173791 A1
(43) Pub. Date: Jul. 26, 2007

(54) System for ophthalmic laser surgery

(75) Inventor: Ferenc Raksi, Irvine, CA (US)

Correspondence Address: CONNOLLY BOVE LODGE & HUTZ LLP, P.O. BOX 2207, WILMINGTON, DE, 19899, US

(73) Assignee: INTRALASE CORP.

(21) Appl. No.: 11/336,660

(22) Filed: Jan. 20, 2006

(51) Int. Cl. A61B 18/18

(52) U.S. Cl. 606/4

(57) ABSTRACT

A system for ophthalmic laser surgery is disclosed. A laser source is adapted for performing ophthalmic laser surgery. A surgical tip is adapted to transmit light from the laser source toward an eye. A reference window is affixed to the surgical tip at a fixed position relative to the laser source. A patient interface is adapted to couple to the eye and to the surgical tip. An applanation lens is coupled to the patient interface. An optical sensor is adapted to detect interference generated between light reflected off the reference window and light reflected off the applanation lens during a coupling procedure between the surgical tip and the patient interface.

### BACKGROUND OF THE INVENTION

1. Field of the Invention

The field of the present invention is systems for ophthalmic laser surgery.

2. Background

Current systems for ophthalmic laser surgery employ a patient interface to couple the surgical tip of the surgical laser with the patient's eye. The patient interface is used so that the surgical tip does not come into direct contact with the eye during a surgical procedure. The surgical tip would require sterilization after each surgical procedure if such direct contact were to occur. A disposable patient interface alleviates the need for repeated sterilization of the equipment used for such surgical procedures.

The patient interface (instead of the surgical tip) includes an applanation lens, which according to U.S. Pat. No. 5,549,632, the disclosure of which is incorporated herein by reference, serves three primary purposes. The first purpose of the applanation lens is to provide a positional reference for the surgical laser; the second is to control the shape of the cornea during the surgical procedure; and the third is to provide a controllable boundary between the epithelium and air in order to reduce the distortion of the surgical laser beam. While all three purposes can be important for the type of ophthalmic laser surgery described, the first purpose can add significant expense to surgical systems because of the precision required for establishing the relative positions between the surgical laser and the applanation lens.

The available options for positioning the surgical laser relative to the applanation lens with the necessary precision are currently limited. Mechanical sensors may be used to sense the physical position of the of the surgical laser and the applanation lens, but such sensors tend to have too low of an accuracy or too high of a cost. Another option is described in U.S. Patent Application Publication No. 20040070761.

The accuracy of focal positioning and reliability and safety is important in surgical applications. For this reason, closed-loop positioning systems have been developed. For example, the system described in U.S. Pat. No. 6,751,033, the disclosure of which is incorporated herein by reference, uses feedback signal from the actual position of the focusing lens assembly to verify system performance, to modify commanded position coordinates, and to warn the user about possible errors. Such systems, however, are not ideal for all circumstances.

### SUMMARY OF THE INVENTION

The present invention is directed towards a system and method for ophthalmic laser surgery. The surgical system includes a laser source which is adapted for performing the ophthalmic surgery and a surgical tip which transmits light from the laser source. A reference window is affixed to the surgical tip at a fixed position relative to the laser source. A patient interface is adapted to couple between the patient's eye and the surgical tip. The patient interface is also coupled to an applanation lens. An optical sensor detects interference in light reflected off the reference window during a coupling procedure between the surgical tip and the patient interface.

In a first separate aspect of the present invention, the optical sensor detects interference generated between light reflected off the reference window and light reflected off the applanation lens. The detected interference may be displayed on a monitor for an operator or may utilized by the system for further processing.

In a second separate aspect of the present invention, the optical sensor determines the distance between the reference window and the applanation lens using the interference. This determination can be made by performing a Fourier transform on the detection signal.

In a third separate aspect of the present invention, which builds on the second separate aspect, the optical sensor determines the distance between the reference window and the applanation lens at a point along the optical axis of the laser source.

In a fourth separate aspect of the present invention, which builds on the second separate aspect, the optical sensor determines the distance between the reference window and the applanation lens at multiple points about the surface of the reference window.

In a fifth separate aspect of the present invention, the optical sensor determines the tilt of the reference window, relative to the applanation lens, using the interference.

In a sixth separate aspect of the present invention, the optical sensor comprises a light source, a photo detector, and a processor. The light source directs light toward the reference window. The interference is generated as this light is reflected off both the reference window and the applanation lens. The photo detector detects the interference and, in response, outputs a detection signal to the processor, which analyzes the detection signal.

In an seventh separate aspect of the present invention, the interference may be generated in any appropriate spectrum of light. For example, the interference may be generated within light outside the visible spectrum, in light having a spectrum above approximately 750 nm, in light having a spectrum that does not include the spectrum of light at which the laser source operates.

In an eighth separate aspect of the present invention, position and tilt information gathered by the optical sensor is employed as part of a closed-loop system to verify or correct focal position commands to the focusing assembly.

In a ninth separate aspect of the present invention, any of the foregoing aspects may be employed in combination.

Accordingly, an improved system and method for ophthalmic laser surgery is disclosed. Other objects and advantages will appear hereinafter.

### BRIEF DESCRIPTION OF THE DRAWINGS

In the drawings, wherein like reference numerals refer to similar components:

FIG. 1 is an exploded perspective view of a patient interface device;

FIG. 2 is a cross-sectional detailed view of the applanation end of a patient interface device;

FIG. 3 is a cross-sectional view of a patient interface device as employed to interface between the surgical tip of an ophthalmic surgical laser system and an eye;

FIG. 4 schematically illustrates an ophthalmic surgical laser system;

FIG. 5A is a graph illustrating a detection signal resulting when the patient interface is disposed outside of coherence range with the reference window;

FIG. 5B is a graph illustrating the Fourier transform of the detection signal of FIG. 5A;

FIG. 6A is a graph illustrating a detection signal resulting when the patient interface is disposed approximately 18 μm from the reference window;

FIG. 6B is a graph illustrating the Fourier transform of the detection signal of FIG. 6A;

FIG. 7A is a graph illustrating a detection signal resulting when the patient interface is disposed approximately 7.5 μm from the reference window; and

FIG. 7B is a graph illustrating the Fourier transform of the detection signal of FIG. 7A.

### DETAILED DESCRIPTION OF THE PREFERRED EMBODIMENTS

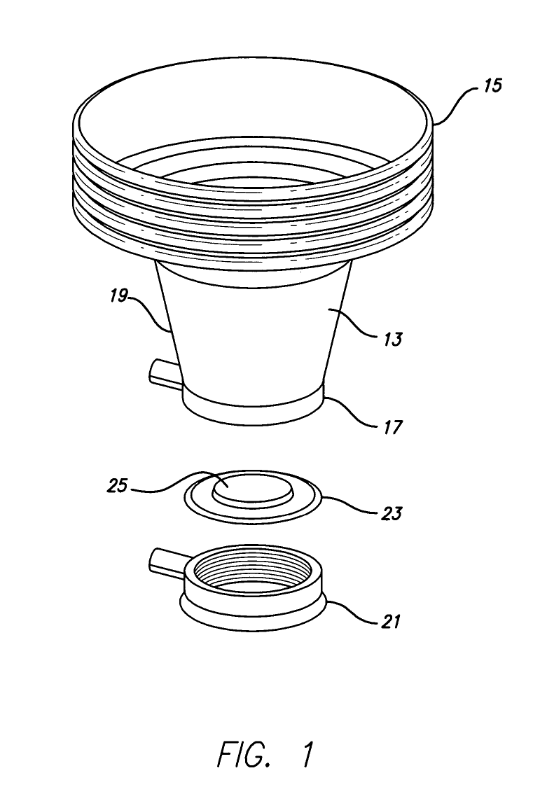

Turning in detail to the drawings, FIG. 1 illustrates a patient interface 11 which is part of a system for performing ophthalmic laser surgery. As described below, the patient interface 11 interfaces between the surgical laser and the cornea of the patient's eye. The frame 13 of the patient interface 11 has an attachment end 15 and an applanation end 17. The attachment end 15 is broad and open to accommodate the exit aperture of an ophthalmic surgical laser system, while the applanation end 17 is considerably narrower to facilitate the coupling between the patient interface 11 and the eye during a surgical procedure. Between the attachment end 15 and the applanation end 17, the sidewalls 19 of the frame 13 form a conical cavity. The shape of the frame 13, however, is generally a matter of design choice. The frame 13 also has non-perforated sidewalls 19. The lack of perforations in the sidewalls 19 helps reduce the chances of cross contamination between the eye and the ophthalmic surgical laser system during a surgical procedure. However, a more open frame may be suitable if sidewall perforations are located such that the sterile barrier is maintained between the eye and the ophthalmic surgical laser system.

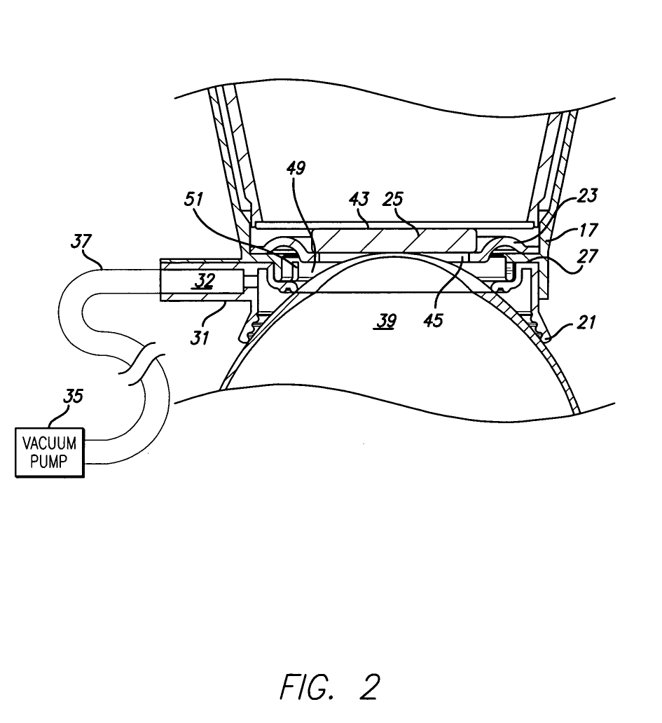

An annular skirt 21, an annular flexible support 23, and a lens 25 are affixed to the applanation end 17 of the frame 13, with the skirt 21 and the flexible support 23 being affixed directly to the frame 13, and the lens 25 being affixed directly to the flexible support 23. The details of the applanation end 17 of the frame 13 are illustrated in FIG. 2. The annular skirt 21 is seated in a complimentary annular channel 27 in the applanation end 17 of the frame 13. The channel 27 includes a side opening 29 through which an arm 31 extends from the skirt 21. The arm 31 houses a passageway 33 which may be affixed to a vacuum pump 35 through a tube 37. The vacuum pump 35 may be a syringe or any other mechanical device capable of generating negative pressure. The patient interface 11 is employed to immobilize the eye during surgery. For this purpose, the skirt 21 is preferably constructed of a soft, pliable material. When the skirt 21 is placed against the eye 39, a chamber 41 is formed and the vacuum pump 35 may be used to create at least a partial vacuum within the chamber, thereby coupling the skirt 21, and thus the patient interface 11, to the eye 39.

The skirt 21 is preferably affixed to the applanation end 17 of the frame 13 using an adhesive which is appropriate for the materials used. Such an adhesive should be one that will not quickly deteriorate when exposed to light from lasers generally employed in ophthalmic surgical laser systems.

The lens 25 has a posterior surface 43 and an anterior surface 45, and may be planar, as shown, or one or both of the surfaces may be curved. The outer edge of the anterior surface 45 is adhered to the flexible support 23. Again, the adhesive may be any that is appropriate for the materials used, with consideration for the laser light to which the adhesive will be exposed. As is understood in the relevant art, the anterior surface 45 of the lens 25 makes contact with the cornea during the surgical procedure and flattens, configures, or otherwise shapes the cornea for the surgical procedure. The geometrical configuration of the lens 25 therefore depends upon the shape to which the cornea is to be conformed during the surgical procedure. The lens 25 is preferably made of a inexpensive high strength transparent material, such as glass, plastic, or the like.

The flexible support 23 is itself adhered to the applanation end 17 of the frame 13 using an adhesive, although a mechanical coupling could also be used. The considerations for the adhesive are again the same.

During ophthalmic laser surgery, a secondary chamber 49 is created when the patient interface 11 is coupled to the eye 39. The secondary chamber 49 is formed by the anterior surface 45 of the lens 25, the flexible support 23, the annular channel 27 of the frame 13, the annular skirt 21, and the cornea of the eye 39. The volume of the secondary chamber 49 changes as the lens 25 moves on the flexible support. The amount of lens movement is an important factor in determining the amount by which the cornea is flattened, configured, or otherwise shaped for the surgical procedure. As the volume of the secondary chamber 49 changes, a localized change of pressure occurs within the pocket. This pressure change can negatively affect the ability to shape the cornea as desired using the lens 25. To alleviate this problem, vent ports 51 are disposed within the applanation end 17 of the frame 13. The vent ports 51 permit the relative pressure of air or fluids within the secondary chamber 49 to equalize to atmospheric pressure. The vent ports 51 preferably do not compromise the sterile barrier between the eye 39 and the ophthalmic surgical laser system, nor do they compromise the established pressure within the vacuum chamber 41. The patient interface 11 may include a single vent port, or up to twelve or more vent ports. Multiple vent ports are preferably regularly spaced in a ring about an axis perpendicular to the lens 25. The vent ports 51 help ensure that the shape of the cornea is dictated solely by pressure upon the posterior surface 43 of the lens 25.

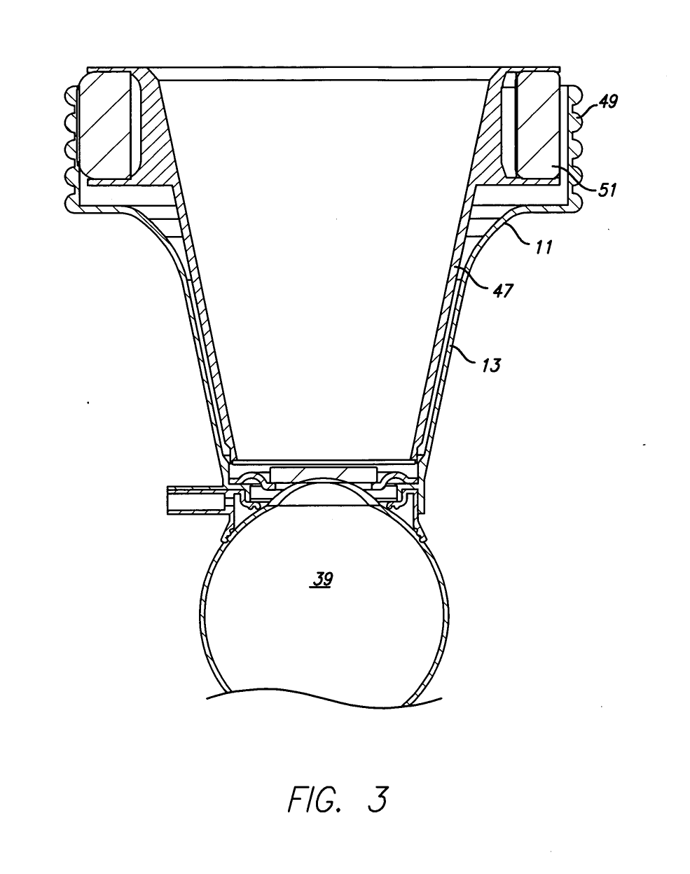

FIG. 3 illustrates the patient interface 11 providing an interface between the patient's eye 39 and the surgical tip 47 of the ophthalmic surgical laser system. The frame 13 of the patient interface 11 is configured to have a complimentary shape to the surgical tip 47. This allows the surgical tip 47 to be inserted directly into the frame 13 and be positioned immediately adjacent the eye 39 without being in physical contact with the eye 39, thereby facilitating the surgical procedure while reducing opportunities for cross contamination between the eye and the surgical tip 47.

The attachment end 15 of the frame 13 is coupled to the surgical tip 47 to further reduce opportunities for cross contamination and to stabilize the interface. This coupling may be achieved by inclusion of a ferromagnetic material in rings 49 circumscribing the attachment end 15 of the frame 13 and complimentary sliding electromagnets 51 in the exit aperture housing 47. The electromagnets 51 are slidable in a radial direction so that when activated, they may couple with, and seal against the attachment end 15 of the frame 13. Alternative methods of coupling the frame 13 to the exit aperture housing 47 may also be employed, including one or more mechanical latches, an inflatable bladder, and the like.

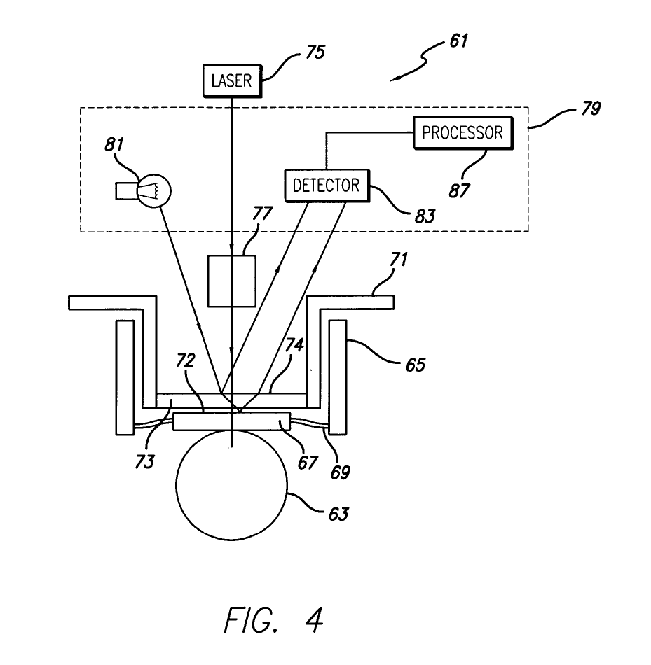

FIG. 4 illustrates an ophthalmic surgical system 61 for performing ophthalmic laser surgery on an eye 63. The patient interface 65 is affixed to the eye in the manner described above, such that the applanation lens 67, which is coupled to the patient interface 65 via the flexible support 69, is in contact with the cornea. The surgical tip 71 if the system is coupled to the patient interface 65 in the manner described above and includes a reference window 73 which is disposed adjacent the applanation lens 67. The laser 75, in combination with the scanning and focusing optics 77, directs the laser beam toward the eye 63 for the surgical procedure. The laser 75 is in a fixed position relative to the reference window 73, insofar as the path of the laser beam between the point of emission from the laser 75 to the reference window 73 has a known length once the system is calibrated. By having the laser 75 in a fixed position relative to the anterior surface 74 of the reference window 73, the laser beam may quickly and easily be focused at the anterior surface 74 of the reference window 73, thus eliminating potentially time consuming alignment procedures. The laser 75 may be of the type described in U.S. Pat. No. 4,764,930 and preferably produces an ultra-short pulsed beam as described in U.S. Pat. No. 5,984,916, the disclosures of which are incorporated herein by reference. The scanning and focusing optics 77 are preferably of the type disclosed in copending U.S. patent application No. ______ filed on ______ in the name of Ferenc Raksi entitled “Laser Scanner”, the disclosure of which is incorporated herein by reference.

The ophthalmic surgical system 61 also includes an optical sensor 79 for determining the proximity of the reference window 73 to the applanation lens 67. A light source 81 directs light toward the reference window 73, and a detector 83 receives light reflected off the posterior surface of the reference window 73, preferably from a point on the reference window 73 which lies on the optical axis of the beam from the surgical laser. Not all light from the light source 81, however, is reflected by the reference window 73. Some of the light passes through the reference window and is reflected off the anterior surface 72 of the applanation lens 67. Light reflected off the applanation lens 67 combines and interferes with light reflected off the reference window 73. This interference creates spatial and spectral modulation in the reflected light which is sensed by the detector 83. In response to detected light, the detector 83 outputs a detection signal which is analyzed by the programmable processor 87 as described below. The spatial modulation pattern and the period of the spectral modulation are used to determine the position of the reference window 73 relative to the anterior surface 72 of the applanation lens 67. It is particularly beneficial to know the relative positions of the reference window 73 and the applanation lens 67 during and following the coupling procedure between the patient interface 65 and the surgical tip 71, so that the surgical laser can be accurately focused within the cornea during the surgical procedure.

The light source 81 may be of any type and may emit light in any spectrum, but it preferably emits a broadband spectrum, on the order of 100 nm or more in bandwidth. Since the coherence length is inversely proportional to the bandwidth of the illuminating light, by appropriately choosing the bandwidth, the coherence length can be made to match acceptance criteria of position tolerance for the applanation lens 67 relative to the reference window 73. In this manner, the presence or the lack of interference creates a pass/fail criteria for the reference window 73 being within a predetermined distance of the applanation lens 67.

In general, no observed modulation means that the two surfaces are further apart than the coherence length of the illuminating light. This distance approximately a few micrometers for light with a 100 nm bandwidth. As an option, the light source may be operated to have a coherence length that is substantially equal to the positioning tolerance between the reference window 73 and the applanation lens 67. In the simplest implementation, the operator is able to visually observe spatial coherence fringes in the detection signal as displayed on a monitor. This allows the operator to determine whether the position of the surgical tip is within a predetermined position tolerance with respect to the patient interface. Manually detecting spatial modulation is more appropriate for visual observation, although spectral modulation is also possible for a better trained observer.

More accurate, quantitative evaluations of the position within the coherence range can be obtained using the detector 83 and programmable processor 87 in combination. Such a combination may be employed to provide more accurate position information to the operator, or alternatively, the combination may. be employed as part of a closed-loop feedback system to automate the positioning process. The following figures illustrate that the detection signal may be advantageously utilized to position the surgical tip relative to the patient interface.

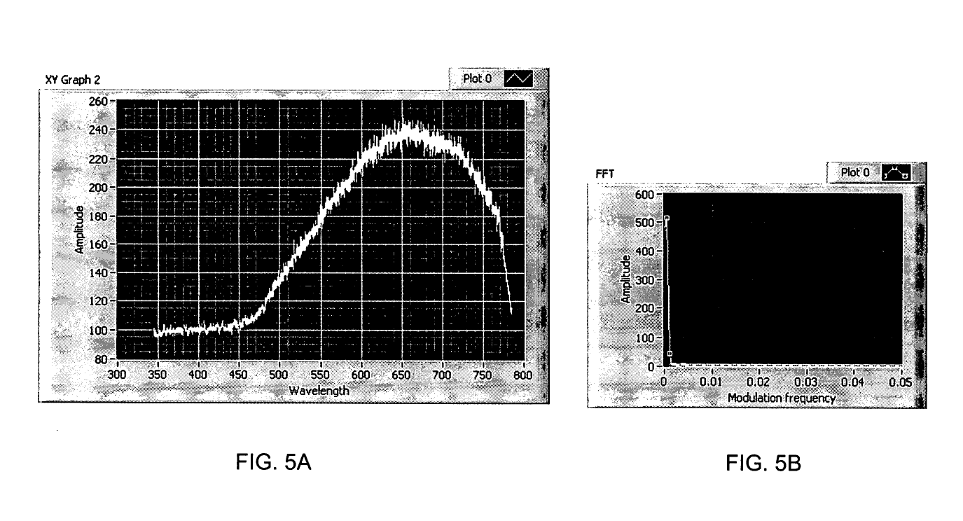

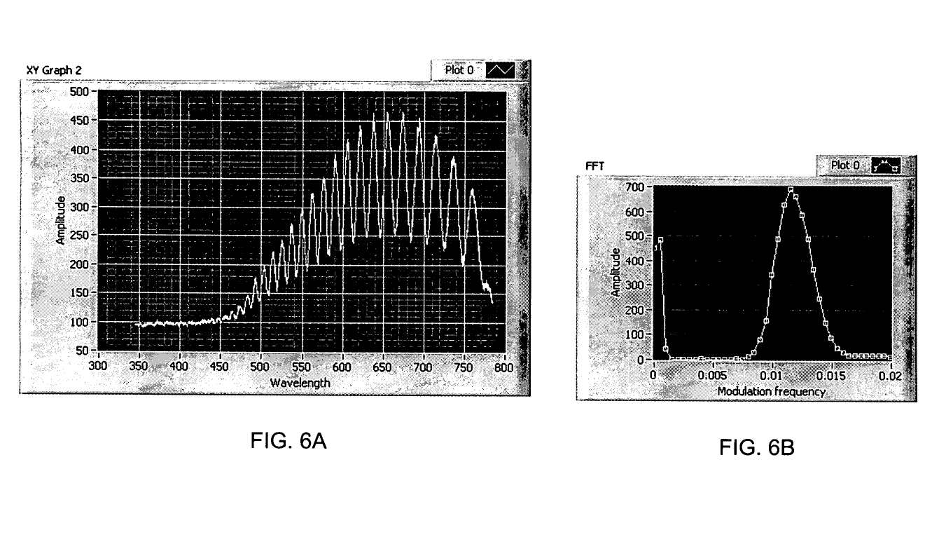

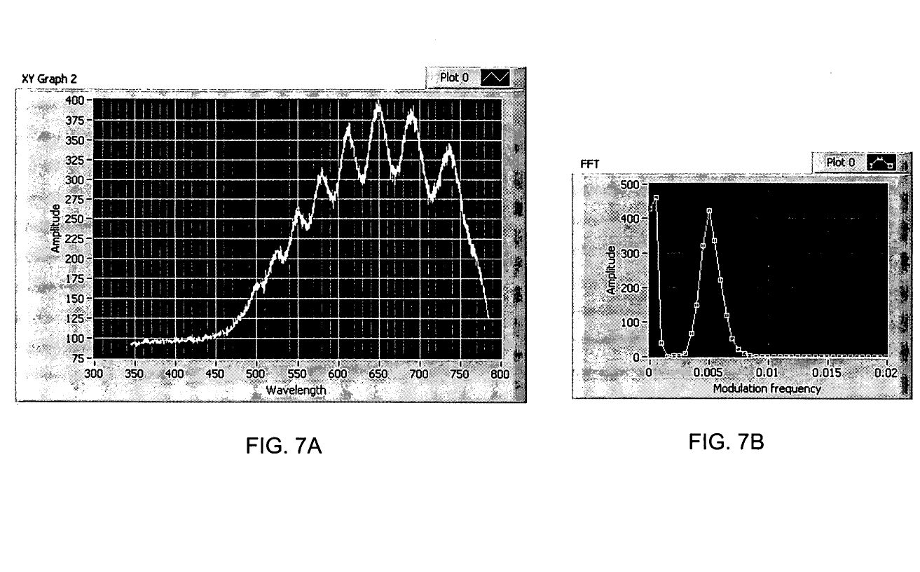

FIG. 5A is a graph showing the spectrum of detected light reflected from the reference window 73 and the applanation lens 67. A Fourier transform of this signal, as seen in FIG. 5B, illustrates that there is no signal at higher modulation frequencies, i.e., there is no peak that does not substantially envelope the zero point on the graph. In this instance, the distance between the reference window 73 and the applanation lens 67 is greater than the coherence range of the illuminating light. FIG. 6A is a graph showing the spectrum of detected light when the distance between the reference window 73 and the applanation lens 67 is 18 μm. The Fourier transform of this signal, shown in FIG. 6B, shows a single higher modulation frequency peak. FIG. 7A is another graph showing the spectrum of detected light when the distance between the reference window 73 and the applanation lens 67 is 7.5 μm. The Fourier transform of this signal, shown in FIG. 7B, also shows a single higher modulation frequency peak. Comparison of the modulation frequency peaks in FIGS. 6B and 7B shows that the peaks are in different locations.

In short, the modulation period, and-therefore the modulation frequency peak, has a direct relationship with the distance between the reference window 73 and the applanation lens 67. Thus, for any particular ophthalmic laser surgery system design, the placement of the modulation frequency peaks can be used to determine the distance between the reference window 73 and the applanation lens 67 during or following the coupling procedure.

The same technique could be employed using spatial modulations of light intensity within the detected light, as opposed to the spectral modulations described above.

The system described above employs a basic interferometer to detect spectral modulations in light reflected from the two surfaces. Those skilled in the art will recognize that other types of interferometers, such as a Michelson interferometer, a Mach-Zender interferometer, a Fabry-Perot interferometer, and the like, may also be employed to the same effect. Also, light from the light source may be allowed to travel through free space, as is illustrated in FIG. 4, or a light guide may be employed as desired. One possible type of light guide is an optical fiber.

Light from the light source may be continuous, pulsed, or temporally modulated as desired. To reduce the effect of background light on the interferometer, such background light arising from room light or scattered light from the laser, the light source can be modulated, chopped, or pulsed. A modulated light source, combined with a gated or modulated detection scheme, such as box-car or lock-in technique, can be employed to significantly improve detection and the signal-to noise ratio.

Another technique that may be employed to filter out background light is proper selection of the spectral band. This may be accomplished at the light source, by filtering out unwanted spectrum, or at the detector by only detecting light in a specified spectrum. For example, the detector may be configured to detect light between 750 nm and 1050 nm. Generally, room light, patient illumination lights, or the light of the surgical laser will lie outside of this spectrum. Blocking light outside of the desired spectrum, by use appropriate filters, or conversely detecting only light within the desired spectrum, effectively filters out unwanted background noise. When selecting the operating spectrum, it is desirable to operate within the spectral sensitivity range of common silicon detectors such as CCD cameras, photodiodes, or photodiode arrays. In general, however, the light source may emit light within any spectrum ranging from the visible range, the infrared range, or beyond.

The above discussion focuses on using a single measurement to determine the spatial relationship along the optical axis of the surgical laser between the reference window 73 and the applanation lens 67. While this information is important, as it directly relates to position of the laser focus in the eye and the cutting depth in the eye, the system may also be used for measurements at multiple locations about the surface of the reference window 73. Such multiple measurements can be used to provide angular tilt information. Using multiple measurements, it is possible to determine the exact position of the reference window 73, with respect to the applanation lens 67, for all six degrees of freedom for a rigid body.

Thus, a system and method for ophthalmic laser surgery are disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A system for ophthalmic laser surgery, the system comprising:
a laser source adapted for ophthalmic laser surgery;
a surgical tip adapted to transmit light from the laser source;
a reference window affixed to the surgical tip at a fixed position relative to the laser source;
a patient interface adapted to couple between an eye and the surgical tip;
an applanation lens coupled to the patient interface; and
an optical sensor adapted to detect interference generated between light reflected off the reference window and light reflected off the applanation lens during a coupling procedure between the surgical tip and the patient interface.

2. The system of claim 1, wherein the optical sensor is further adapted to determine a distance between the reference window and the applanation lens based on the detection signal.

3. The system of claim 2, wherein the distance is measured at a point along an optical axis of light from the laser source.

4. The system of claim 2, wherein the distance is measured at multiple points about the reference window.

5. The system of claim 1, wherein the optical sensor is further adapted to determine a tilt of the reference window relative to the applanation lens.

6. The system of claim 1, wherein the optical sensor comprises:
a light source adapted to direct light toward the reference window;
a photo detector adapted to detect the interference and output a detection signal in response to the detected interference; and
a processor adapted to analyze the detection signal.

7. The system of claim 5, wherein the processor is further adapted to perform a Fourier transform on the detection signal.

8. The system of claim 1, wherein the optical sensor is adapted to provide closed-loop feedback for positioning the surgical tip relative to the applanation lens.

9. The system of claim 1, wherein the interference is generated from light having a spectrum outside of a visible spectrum.

10. The system of claim 1, wherein the interference is generated from light having a spectrum above approximately 750 nm.

11. The system of claim 1, wherein the interference is generated from light having a coherence length approximately equal to a position tolerance between the reference window and the applanation lens.

12. The system of claim 1, wherein the interference is generated from light having a first spectrum which does not include light within a second spectrum.

13. The system of claim 12, wherein the laser source emits light in the second spectrum.

14. A system for ophthalmic laser surgery, the system comprising:
a laser source adapted for ophthalmic laser surgery;
a surgical tip adapted to transmit light from the laser source;
a reference window affixed to the surgical tip at a fixed position relative to the laser source;
a patient interface adapted to couple between an eye and the surgical tip;
an applanation lens coupled to the patient interface;
a light source adapted to direct light toward the reference window;
a photo detector adapted to detect interference generated between light reflected off the reference window and light reflected off the applanation lens during a coupling procedure between the surgical tip and the patient interface, wherein the photo detector outputs a detection signal in response to the detected interference; and
a processor adapted to analyze the detection signal.

15. The system of claim 14, wherein the processor is further adapted to determine a distance between the reference window and the applanation lens based on the detection signal.

16. The system of claim 15, wherein the distance is measured at a point along an optical axis of light from the laser source.

17. The system of claim 15, wherein the distance is measured at multiple points about the reference window.

18. The system of claim 14, wherein the processor is further adapted to provide closed-loop feedback for positioning the surgical tip relative to the applanation lens.

19. The system of claim 14, wherein the processor is further adapted to perform a Fourier transform on the detection signal.

20. The system of claim 14, wherein the processor is further adapted to determine a tilt of the reference window relative to the applanation lens.

21. The system of claim 14, wherein the interference is generated from light having a spectrum outside of a visible spectrum.

22. The system of claim 14, wherein the interference is generated from light having a spectrum above approximately 750 nm.

23. The system of claim 14, wherein the interference is generated from light having a coherence length approximately equal to a position tolerance between the reference window and the applanation lens.

24. The system of claim 14, wherein the interference is generated from light having a first spectrum which does not include light within a second spectrum.

25. The system of claim 24, wherein the laser source emits light within the second spectrum.

26. A system for ophthalmic laser surgery, the system comprising:
a laser source adapted for ophthalmic laser surgery;
a surgical tip adapted to transmit light from the laser source;
a reference window affixed to the surgical tip at a fixed position relative to the laser source;
a patient interface adapted to couple between an eye and the surgical tip;
an applanation lens coupled to the patient interface; and
means to detect interference generated between light reflected off the reference window and light reflected off the applanation lens during a coupling procedure between the surgical tip and the patient interface.

27. The system of claim 26, wherein the means to detect the interference outputs a detection signal in response to detected interference.

28. The system of claim 27 further comprising a processor adapted to analyze the detection signal.

29. The system of claim 28, wherein the processor is further adapted to determine a distance between the reference window and the applanation lens based on the detection signal.

30. The system of claim 29, wherein the distance is measured at a point along an optical axis of light from the laser source.

31. The system of claim 29, wherein the distance is measured at multiple points about the reference window.

32. The system of claim 28, wherein the processor is further adapted to provide closed-loop feedback for positioning the surgical tip relative to the applanation lens.

33. The system of claim 28, wherein the processor is further adapted to perform a Fourier transform on the detection signal.

34. The system of claim 28, wherein the processor is further adapted to determine a tilt of the reference window relative to the applanation lens.

35. The system of claim 26, wherein the interference is generated from light outside of a visible spectrum.

36. The system of claim 26, wherein the interference is generated from light having a wavelength above approximately 750 nm.

37. The system of claim 26, wherein the interference is generated from light having a coherence length approximately equal to a position tolerance between the reference window and the applanation lens.

38. The system of claim 26, wherein the interference is generated from light having a first spectrum which does not include light within a second spectrum.

39. The system of claim 38, wherein the laser source emits light within the second spectrum.

40. A method of ophthalmic surgery, the method comprising:
placing a laser source at a fixed position relative to a reference window, wherein the reference window is affixed to a surgical tip and light from the laser source is transmitted through the surgical tip;
coupling a patient interface to a cornea, wherein the patient interface is adapted to couple with the surgical tip and is coupled to an applanation lens;
moving the surgical tip into position for coupling with the patient interface; and
detecting interference as the surgical tip is moved into position for coupling with the patient interface, wherein the interference is generated between light reflected off the reference window and light reflected off the applanation lens.

41. The method of claim 40 further comprising providing closed-loop feedback information for moving the surgical tip into position for coupling with the patient interface.

42. The method of claim 40, wherein detecting the interference includes:
directing light from a light source toward the reference window;
outputting a detection signal in response to the detected interference; and
analyzing the detection signal.

43. The method of claim 42, wherein directing light from a light source includes selecting a bandwidth of light having a coherence length approximately equal to a position tolerance between the reference window and the applanation lens.

44. The method of claim 42, wherein analyzing the detection signal includes performing a Fourier transform on the detection signal.

45. The method of claim 40 further comprising determining a distance between the reference window and the applanation lens based on the interference.

46. The method of claim 45, wherein determining the distance includes determining the distance at a point along an optical axis of light from the laser source.

47. The method of claim 45, wherein determining the distance includes determining the distance at multiple points about the reference window.

48. The method of claim 40 further comprising determining a tilt of the reference window relative to the applanation lens using the interference.

49. The method of claim 40, wherein detecting the interference includes detecting the interference in light having a spectrum outside of a visible spectrum.

50. The method of claim 40, wherein detecting the interference includes detecting the interference in light having a wavelength above approximately 750 nm.

51. The method of claim 40, wherein detecting the interference includes detecting the interference in light having a first spectrum which does not include light within a second spectrum.

52. The method of claim 51, wherein the laser source emits light within the second spectrum.

53. A method of ophthalmic surgery, the method comprising:
placing a laser source at a fixed position relative to a reference window, wherein the reference window is affixed to a surgical tip and light from the laser source is transmitted through the surgical tip;
coupling a patient interface to a cornea, wherein the patient interface is adapted to couple with the surgical tip and is coupled to an applanation lens;
moving the surgical tip into position for coupling with the patient interface; and
directing light from a light source toward the reference window as the surgical tip is moved into position for coupling with the patient interface;
detecting interference generated between light reflected off the reference window and light reflected off the applanation lens;
outputting a detection signal in response to the detected interference; and
analyzing the detection signal.

54. The method of claim 53 further comprising providing closed-loop feedback information for moving the surgical tip into position for coupling with the patient interface.

55. The method of claim 53, wherein directing light from a light source includes selecting a bandwidth of light having a coherence length approximately equal to a position tolerance between the reference window and the applanation lens.

56. The method of claim 53, wherein analyzing the detection signal includes performing a Fourier transform on the detection signal.

57. The method of claim 53, wherein analyzing the detection signal includes determining a distance between the reference window and the applanation lens based on the interference.

58. The method of claim 57, wherein determining the distance includes determining the distance at a point along an optical axis of light from the laser source.

59. The method of claim 57, wherein determining the distance includes determining the distance at multiple points about the reference window.

60. The method of claim 53, wherein analyzing the detection signal includes determining a tilt of the reference window relative to the applanation lens using the interference.

61. The method of claim 53, wherein detecting the interference includes detecting the interference in light having a spectrum outside of a visible spectrum.

62. The method of claim 53, wherein detecting the interference includes detecting the interference in light having a wavelength above approximately 750 nm.

63. The method of claim 53, wherein detecting the interference includes detecting the interference in light having a first spectrum which does not include light within a second spectrum.

64. The method of claim 63, wherein the laser source emits light within the second spectrum.
